(19) United States
(12) Patent Application Publication
(10) Pub. No.: US 2025/0164509 A1
(43) Pub. Date: May 22, 2025

(54) METHODS AND RELATED ASPECTS FOR DETECTING AND QUANTIFYING AMOUNTS OF N-TERMINAL PROHORMONE B-TYPE NATRIURETIC PEPTIDE IN WHOLE BLOOD SAMPLES

(71) Applicant: ARIZONA BOARD OF REGENTS ON BEHALF OF ARIZONA STATE UNIVERSITY, Scottsdale, AZ (US)

(72) Inventors: Shaopeng WANG, Chandler, AZ (US); Chao CHEN, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/949,000

(22) Filed: Nov. 15, 2024

Related U.S. Application Data
(60) Provisional application No. 63/600,159, filed on Nov. 17, 2023.

(51) Int. Cl. G01N 33/74; B01L 3/00; G01N 33/543

(52) U.S. Cl. CPC G01N 33/74; B01L 3/502761; G01N 33/54346; G01N 33/54386; B01L 2200/0647; B01L 2200/16; B01L 2300/0654; B01L 2300/0681; G01N 2333/58; G01N 2470/04

(57) ABSTRACT

Provided herein are methods of quantifying amounts of N-terminal prohormone B-type natriuretic peptide (NT-proBNP) in whole blood samples. The methods include contacting detection antibody-NT-proBNP complexes with a plurality of capture antibodies, or antigen binding portions thereof, that specifically bind to the NT-proBNP molecules of the detection antibody-NT-proBNP complexes to form captured NT-proBNP complexes, and contacting NPs that each comprise a second recognition moiety that binds to the first recognition moiety of the detection antibodies, or antigen binding portions thereof, of the captured NT-proBNP complexes to form captured NP-NT-proBNP complexes. The methods also include taking images of the captured NP-NT-proBNP complexes to produce imaged captured NP-NT-proBNP complexes using a detection mechanism, and quantifying an amount of NT-proBNP in the sample aliquots using the imaged captured NP-NT-proBNP complexes. Additional methods as well as related devices and systems are also provided.

### CROSS-REFERENCE TO RELATED APPLICATIONS

This application claims the benefit of U.S. Provisional Application No. 63/600,159 filed Nov. 17, 2023, the disclosure of which is incorporated herein in its entirety.

### BACKGROUND

With notoriously high prevalence and economic burden, heart failure has posed an enormous challenge to the global healthcare systems. In the United States alone, more than 6 million cases of heart failure have led to around $31 billion expenditure of the health sector. Heart failure is characterized as compromised pumping capability of heart, leading to reduced blood supply or increased blood pressure. In response to cardiac wall stress caused by heart failure, a hormone called B-type natriuretic peptide (BNP) is secreted by the ventricles to help maintain blood pressure. BNP and its biologically inactive counterpart NT-proBNP are produced in equal molar ratio by the cleavage of their precursor prohormone proBNP. Such a strong biological correlation lays the foundation for the use of BNP and NT-proBNP in the diagnosis, prognosis and treatment management of heart failure. With the negative predictive values ranging from 0.94 to 0.98 at the cutoff points of 35 pg/mL for BNP and 125 pg/mL for NT-proBNP, both peptides have been recommended by clinical guidelines as biomarkers for excluding heart failure. However, significantly longer half-life of NT-proBNP than BNP (1-2 h vs 20 min) results in its higher baseline plasma concentration and smaller biological variability, which makes NT-proBNP a better target for in vitro diagnostics.

The biggest challenge for the healthcare of heart failure lies in its chronic nature and high readmission rate of 23%, which entails a very regular monitoring of the patients' status to minimize hospitalization and improve mortality. This highlights the need for a POC testing of biomarkers like NT-proBNP, which could be used outside of clinical lab at clinic or home. Nonetheless, despite many NT-proBNP assays on the market, most of them are bulky central laboratory devices. Available POC assays require large sample volume or trained professionals to collect and process blood samples still confines their use to emergency department and inpatient settings. To tackle the above limitations, various efforts in developing POC biosensors have been reported in the literature for NT-proBNP. Many of them adopt lateral flow assay (LFA). LFA has the merits of ease of development, no sample preparation and low cost. The downside is that the sensitivity or precision usually falls short of clinical requirement. In addition, their measuring range is too narrow compared to clinically relevant NT-proBNP range. POC surface plasmon resonance (SPR) biosensor is also implemented for the detection of NT-proBNP. Other than traditional gold surface, a silicon dioxide chip was examined to reduce required sample volume. These SPR-based biosensors, however, still don't satisfy the sensitivity requirement for clinical testing because of their higher than needed ng/mL level limit of detection (LoD). Another approach is a field effect transistor (FET) biosensor. Others have developed an integrated POC device for detecting NT-proBNP based on FET sensors. This integrated system is capable of measuring NT-proBNP in the range of 1-10,000 pg/mL from just 4 μL serum/plasma sample in 5 minutes. Unfortunately, it consists of 2 large structural units and is incompatible with whole blood. To achieve whole blood compatibility, others created a handheld sensor and used gravity sedimentation to separate plasma by inverting the sensor after applying blood. Nevertheless, the repeatability isn't good enough, with huge variation from sensor to sensor. Another work devised a microfluidics-based electrochemical sensor with all the reagents dried inside the microfluidic channel, which enables no reagent handling. However, the 570 pg/mL LoD and the incompatibility with whole blood substantially compromises its usefulness. These reported biosensors in development have their own advantages but they are, more or less, still limited by insufficient sensitivity or precision, low dynamic range, large size, or complicated sample preparation requirement.

Accordingly, there is a need for additional methods for detecting and quantifying NT-proBNP in whole blood samples.

### SUMMARY

This present disclosure provides, in some aspects, a point-of-care diagnostic system for detecting, quantifying, and monitoring the biomarker N-terminal proBNP (NT-proBNP) in patients being assessed for possible heart failure. In some embodiments, for example, the present disclosure provides a digital counting-based microfluidic nanobiosensor that can quantify NT-proBNP concentration in whole blood samples with a point-of-care (POC) compatible protocol. In some embodiments, the integrated microfluidic device includes a control circuit that automatically runs the digital NT-proBNP immunoassay. In some embodiments, an NT-proBNP-containing whole blood sample mixed with biotinylated detection antibody is passed through a plasma separator and reacts with a capture antibody-functionalized sensor surface. In some embodiments, streptavidin coated gold nanoparticles (GNPs) are then released to mark the surface-bound single NT-proBNP immunocomplexes and recorded with a bright field microscopy image. In some embodiments, the number of captured GNPs is quantified by a digital counting algorithm and correlated to the NT-proBNP concentration in the sample, for example, via a calibration curve. In some embodiments, the assays and devices disclosed herein achieve NT-proBNP detection in the range of about 50-10,000 pg/mL from only about 7 μL of a whole blood sample in about 10 minutes or less. These and other attributes of the present disclosure will be apparent upon a complete review of the specification, including the accompanying figures.

According to various embodiments, a method of quantifying an amount of N-terminal prohormone B-type natriuretic peptide (NT-proBNP) in a whole blood sample is presented. The method includes contacting detection antibody-NT-proBNP complexes with a plurality of capture antibodies, or antigen binding portions thereof, that specifically bind to the NT-proBNP molecules of the detection antibody-NT-proBNP complexes to form captured NT-proBNP complexes, wherein the detection antibody-NT-proBNP complexes were formed by contacting a substantially unprocessed NT-proBNP molecule-containing whole blood sample aliquot with a plurality of detection antibodies, or antigen binding portions thereof, that each comprise a first recognition moiety, which detection antibodies, or antigen binding portions thereof, specifically bind to the NT-proBNP molecules in the substantially unprocessed NT-proBNP molecule-containing whole blood sample aliquot to form the detection antibody-NT-proBNP complexes; contacting nanoparticles (NPs) that each comprise a second recognition moiety that binds to the first recognition moiety of the detection antibodies, or antigen binding portions thereof, of the captured NT-proBNP complexes to form captured NP-NT-proBNP complexes; taking images of the captured NP-NT-proBNP complexes to produce imaged captured NP-NT-proBNP complexes using a detection mechanism; and, quantifying an amount of NT-proBNP in the sample aliquots using the imaged captured NP-NT-proBNP complexes, thereby quantifying the amount of NT-proBNP in the whole blood sample.

Various optional features of the above embodiments include the following. The detection antibodies, or antigen binding portions thereof, specifically bind to a first epitope of the NT-proBNP molecules, wherein the capture antibodies, or antigen binding portions thereof, specifically bind to a first epitope of the NT-proBNP molecules, and wherein the first and second epitopes differ from one another. The captured NP-NT-proBNP complexes each comprise a single bound NP. The quantifying step comprises digitally counting the imaged captured NP-NT-proBNP complexes in the images to quantify the amount of NT-proBNP in the whole blood sample. The quantifying step comprises determining a concentration of the NT-proBNP in the whole blood sample. The first recognition moiety is a compound selected from the group consisting of: biotin, streptavidin, avidin, an antibody, an antigen, an aptamer, a protein, a peptide, and a carbohydrate. The second recognition moiety is a compound selected from the group consisting of: biotin, streptavidin, avidin, an antibody, an antigen, an aptamer, a protein, a peptide, and a carbohydrate. The method includes flowing the detection antibody-NT-proBNP complexes through a plasma separator prior to contacting the detection antibody-NT-proBNP complexes with the plurality of capture antibodies, or antigen binding portions thereof. The plurality of capture antibodies, or antigen binding portions thereof, are disposed on a surface of a solid support. The method includes performing at least a portion of the method in a microfluidic digital nanobiosensor (mDNB) device or system. The method includes obtaining the whole blood sample from a subject. The method includes administering, or discontinuing administering, therapy to the subject based at least in part on the amount of NT-proBNP in the whole blood sample obtained from the subject. The method includes generating a therapy recommendation for the subject based at least in part on the amount of NT-proBNP in the whole blood sample obtained from the subject.

According to various embodiments, a microfluidic digital nanobiosensor (mDNB) device is presented. The mDNB device comprises: a body structure comprising at least one microfluidic channel disposed at least partially in the body structure; a sample inlet area disposed at least partially in the body structure and in fluid communication with the microfluidic channel, wherein the sample inlet area is configured to receive sample aliquots that comprise mixtures of substantially unprocessed N-terminal prohormone B-type natriuretic peptide (NT-proBNP) molecule-containing whole blood and a plurality of detection antibodies, or antigen binding portions thereof, that each comprise a first recognition moiety, which detection antibodies, or antigen binding portions thereof, specifically bind to the NT-proBNP molecules in the whole blood to form detection antibody-NT-proBNP complexes; an assay area disposed at least partially in the body structure and in fluid communication with the microfluidic channel; a plasma separator disposed in the microfluidic channel between the sample inlet area and the assay area; a plurality of capture antibodies, or antigen binding portions thereof, disposed on a surface of the assay area, wherein the capture antibodies, or antigen binding portions thereof, specifically bind to the NT-proBNP molecules of the detection antibody-NT-proBNP complexes when the detection antibody-NT-proBNP complexes are conveyed from the sample inlet area to the assay area through at least a portion of the microfluidic channel through the plasma separator into contact with the plurality of capture antibodies, or antigen binding portions thereof, to form captured NT-proBNP complexes; and, a nanoparticle (NP) reservoir disposed at least partially in the body structure and in fluid communication with the microfluidic channel, which NP reservoir is configured to contain NPs that each comprise a second recognition moiety that binds to the first recognition moiety of the detection antibodies, or antigen binding portions thereof, of the captured NT-proBNP complexes when the NPs are conveyed from the NP reservoir to the assay area through at least a portion of the microfluidic channel into contact with the captured NT-proBNP complexes to form captured NP-NT-proBNP complexes; wherein the mDNB device is configured to operably connect to a fluid conveyance mechanism that effects fluid conveyance through the microfluidic channel to and/or from the sample inlet area, the assay area, and the NP reservoir; and wherein the mDNB device is configured to operably interface with a detection mechanism that images the captured NP-NT-proBNP complexes in the assay area to produce imaged captured NP-NT-proBNP complexes such that a controller operably connected to the detection mechanism quantifies an amount of NT-proBNP in the sample aliquots from the imaged captured NP-NT-proBNP complexes.

Various additional optional features of the above embodiments include the following. The detection antibodies, or antigen binding portions thereof, specifically bind to a first epitope of the NT-proBNP molecules, wherein the capture antibodies, or antigen binding portions thereof, specifically bind to a first epitope of the NT-proBNP molecules, and wherein the first and second epitopes differ from one another. The captured NP-NT-proBNP complexes each comprise a single bound NP. The controller comprises a processor, and a memory communicatively coupled to the processor, the memory storing non-transitory computer executable instructions which, when executed on the processor, perform operations comprising: digitally counting the imaged captured NP-NT-proBNP complexes in the images to quantify the amount of NT-proBNP in the sample aliquots. The amount of NT-proBNP in the sample aliquots comprises a concentration of the NT-proBNP in the sample aliquots. The detection mechanism comprises a bright-field microscope. The NPs comprise metallic nanoparticles (MNPs). A kit comprises the mDNB device. The sample inlet area comprises a sample inlet port. A microfluidic chip or cartridge comprises the mDNB device. A point-of-care device comprises or is configured to receive the mDNB device. The first recognition moiety is a compound selected from the group consisting of: biotin, streptavidin, avidin, an antibody, an antigen, an aptamer, a protein, a peptide, and a carbohydrate. The second recognition moiety is a compound selected from the group consisting of: biotin, streptavidin, avidin, an antibody, an antigen, an aptamer, a protein, a peptide, and a carbohydrate.

According to various embodiments, a microfluidic digital nanobiosensor (mDNB) system is presented. The mDNB system comprises: a mDNB device receiving area structured to receive at least one mDNB device that comprises: a body structure comprising at least one microfluidic channel disposed at least partially in the body structure; a sample inlet area disposed at least partially in the body structure and in fluid communication with the microfluidic channel, wherein the sample inlet area is configured to receive sample aliquots that comprise mixtures of substantially unprocessed N-terminal prohormone B-type natriuretic peptide (NT-proBNP) molecule-containing whole blood and a plurality of detection antibodies, or antigen binding portions thereof, that each comprise a first recognition moiety, which detection antibodies, or antigen binding portions thereof, specifically bind to the NT-proBNP molecules in the whole blood to form detection antibody-NT-proBNP complexes; an assay area disposed at least partially in the body structure and in fluid communication with the microfluidic channel; a plasma separator disposed in the microfluidic channel between the sample inlet area and the assay area; a plurality of capture antibodies, or antigen binding portions thereof, disposed on a surface of the assay area, wherein the capture antibodies, or antigen binding portions thereof, specifically bind to the NT-proBNP molecules of the detection antibody-NT-proBNP complexes when the detection antibody-NT-proBNP complexes are conveyed from the sample inlet area to the assay area through at least a portion of the microfluidic channel through the plasma separator into contact with the plurality of capture antibodies, or antigen binding portions thereof, to form captured NT-proBNP complexes; and, a nanoparticle (NP) reservoir disposed at least partially in the body structure and in fluid communication with the microfluidic channel, which NP reservoir is configured to contain NPs that each comprise a second recognition moiety that binds to the first recognition moiety of the detection antibodies, or antigen binding portions thereof, of the captured NT-proBNP complexes when the NPs are conveyed from the NP reservoir to the assay area through at least a portion of the microfluidic channel into contact with the captured NT-proBNP complexes to form captured NP-NT-proBNP complexes; a fluid conveyance mechanism that operably connects to the mDNB device when the mDNB device is received in the mDNB device receiving area, which fluid conveyance mechanism is configured to effect fluid conveyance through the microfluidic channel to and/or from the sample inlet area, the assay area, and the NP reservoir; a detection mechanism that is configured to take images of the captured NP-NT-proBNP complexes in the assay area to produce imaged captured NP-NT-proBNP complexes when the mDNB device is received in the mDNB device receiving area; and, a controller comprises a processor, and a memory communicatively directly or remotely coupled to the processor, the memory storing non-transitory computer executable instructions which, when executed on the processor, perform operations comprising: conveying the fluid through the microfluidic channel to and/or from the sample inlet area, the assay area, and the NP reservoir using the fluid conveyance mechanism; taking the images of the captured NP-NT-proBNP complexes in the assay area to produce the imaged captured NP-NT-proBNP complexes using the detection mechanism; and quantifying an amount of NT-proBNP in the sample aliquots using the imaged captured NP-NT-proBNP complexes.

Various additional optional features of the above embodiments include the following. The detection antibodies, or antigen binding portions thereof, specifically bind to a first epitope of the NT-proBNP molecules, wherein the capture antibodies, or antigen binding portions thereof, specifically bind to a first epitope of the NT-proBNP molecules, and wherein the first and second epitopes differ from one another. The captured NP-NT-proBNP complexes each comprise a single bound NP. The amount of NT-proBNP in the sample aliquots comprises a concentration of the NT-proBNP in the sample aliquots. The detection mechanism comprises a bright-field microscope. The NPs comprise metallic nanoparticles (MNPs). A microfluidic chip or cartridge comprises the mDNB device. The system comprises a point-of-care device that comprises or is configured to receive the mDNB device. The first recognition moiety is a compound selected from the group consisting of: biotin, streptavidin, avidin, an antibody, an antigen, an aptamer, a protein, a peptide, and a carbohydrate. The second recognition moiety is a compound selected from the group consisting of: biotin, streptavidin, avidin, an antibody, an antigen, an aptamer, a protein, a peptide, and a carbohydrate. The mDNB system is configured to detect the NT-proBNP molecules in a range of about 50-10,000 pg/mL from less than about 10 μL of a given whole blood sample in about 15 minutes or less.

### BRIEF DESCRIPTION OF DRAWINGS

FIG. 1 is a flow chart that schematically shows exemplary method steps quantifying an amount of N-terminal prohormone B-type natriuretic peptide (NT-proBNP) in a whole blood sample according to some aspects disclosed herein.

FIG. 2 is a schematic diagram of an exemplary point-of-care device and microfluidic digital nanobiosensor (mDNB) device suitable for use with certain aspects disclosed herein.

FIG. 3 is a schematic diagram of an exemplary system suitable for use with certain aspects disclosed herein.

FIGS. 4A and 4B. Schematic of working principle of the microfluidic digital nanobiosensor for point-of-care NT-proBNP detection. (a) Experimental setup and assay workflow. Assay zone is functionalized with capture antibody (cAb). Blood sample mixed with biotinylated detection antibody (dAb) is loaded to a plasma separator and transported to the assay zone, allowing the formation of sandwich immunocomplex. Later, streptavidin conjugated GNPs are released from the reservoir to label individual immunocomplexes, which are recorded by bright-field imaging. (b) Signal processing workflow. Bright-field images of GNPs for low concentrations of NT-proBNP are processed with digital counting while those of high concentrations are analyzed by image segmentation, based on which a standard curve is obtained for measuring NT-proBNP concentration in unknown samples.

FIGS. 5A-5C. Overall design of the POC microfluidic device for NT-proBNP detection from whole blood samples. (a) Schematic of the 3 main components of the microfluidic device and their connection. The microfluidic chip serves as the platform for conducting immunoreaction of NT-proBNP. The micro vacuum pump is connected to the microfluidic chip through a solenoid switch valve and a waste reservoir, and provides the driving force for microfluidic flow. A microcontroller board is used to control the micro vacuum pump and the fluid flow in the channel. (b) Photographs of the ready-to-use microfluidic chip, (c) with the two solenoid valves installed.

FIGS. 6A-61. Generation of the calibration curve. Processed bright-field images showing the surface-bound individual GNPs corresponding to (a) horse serum (0 pg/mL), (b) nonspiked pooled normal human plasma (8.24 pg/mL), and (c-f) 50, 500, 2000, and 10 000 pg/mL of NT-proBNP spiked into the human plasma (concentrations in the figure include the 8.2 pg/mL endogenous level). Only a section (1.56%) of the full image is shown for clarity. The scale bar represents 5 μm. The number of GNPs increases with NT-proBNP concentration. (g) Flowchart of ImageJ data processing for hybrid calibration curve generation. (h) Calibration curve of NT-proBNP detection in human plasma for MDIA. (i) Calibration curve of NT-proBNP detection for traditional ELISA. The error bars are standard deviation of triplicates.

FIGS. 7A and 7B. Validation of the MDIA. (a) Recovery of MDIA for 3 different concentrations of NT-proBNP spiked into human whole blood. The spiked concentrations were confirmed by using conventional ELISA to measure their centrifuged plasma. (b) Pearson's correlation between MDIA and Roche's Elecsys proBNP II assay. The error bars are the standard deviation of triplicate tests.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and devices, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a NT-proBNP molecule, e.g., a molecule in which one or more immunoglobulin chains is not full length, but which specifically binds to a NT-proBNP molecule. Examples of binding portions encompassed within the term “antigen-binding portion of an antibody include (i) a Fab fragment, a monovalent fragment consisting of the VLC, VHC, CL and CH1 domains: (ii) a F(ab′)2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV)). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody portions are obtained using conventional techniques known to those with skill in the art, and the portions are screened for utility in the same manner as are intact antibodies.

Binding: As used herein, the term “binding” or “binding interaction”, typically refers to a non-covalent association between or among two or more entities. “Direct” binding involves physical contact between entities or moieties; “indirect” binding involves physical interaction by way of physical contact with one or more intermediate entities. Binding between two or more entities can be assessed in any of a variety of contexts-including where interacting entities or moieties are studied in isolation or in the context of more complex systems (e.g., while covalently or otherwise associated with a carrier entity and/or in a biological system or cell).

Binding Moiety: As used herein, the term “binding moiety” refers to a portion of a chemical compound or structure that selectively or preferentially binds to another chemical compound or structure. In some embodiments, for example, a surface (e.g., a solid surface of a microfluidic cavity or channel) is functionalized or conjugated with binding moieties (e.g., capture antibodies or antigen binding portions thereof), which selectively or preferentially bind to NT-proBNP molecules.

Conjugate: As used herein, “conjugate” refers to a reversible or irreversible connection between two or more substances or components. In some embodiments, for example, gold nanoparticles (AuNPs) or other NPs are connected to recognition moieties. In some embodiments, capture antibodies or antigen binding portions thereof are conjugated to a solid support surface (e.g., a surface disposed in a cavity of a microfluidic cartridge or device). In some embodiments, conjugation is via one or more linker compounds. In some embodiments, recognition moieties are conjugated to detection antibodies or antigen binding portions thereof.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Detect: As used herein, the term “detect,” “detecting,” or “detection” refers to an act of determining the existence or presence of one or more target analytes (e.g., an NT-proBNP molecule in a sample.

Epitope: As used herein, “epitope” refers to the part of an antigen to which an antibody and/or an antigen binding portion binds.

N-terminal prohormone B-type natriuretic peptide: As used herein, the term “N-terminal prohormone B-type natriuretic peptide” or “NT-proBNP” refers to a prohormone with a 76 amino acid N-terminal inactive protein that is cleaved from the molecule to release B-type natriuretic peptide (BNP, also known as brain natriuretic peptide 32).

Recognition moiety: As used herein, the term “recognition moiety” refers to a portion of a chemical compound or structure that selectively or preferentially binds to another chemical compound or structure. In some embodiments, a detection antibody or antigen binding portion thereof includes biotin as a recognition moiety, which selectively or preferentially binds to another recognition moiety, such as streptavidin or avidin bound or conjugated to a nanoparticle (NP).

Sample: As used herein, “sample” or “fluidic sample” refers to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide), which is assayed as described herein. A sample may also be any body fluid or excretion (for example, but not limited to, blood, urine, stool, saliva, tears, bile) that contains cells, cell components, or non-cellular fractions.

Subject: As used herein, “subject” refers to an animal, such as a mammalian species (e.g., human, dog, cat) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). In certain embodiments, the subject is a human. In certain embodiments, the subject is a companion animal, including, but not limited to, a dog or a cat. A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

System: As used herein, “system” in the context of analytical instrumentation refers a group of objects and/or devices that form a network for performing a desired objective.

### DETAILED DESCRIPTION

The notoriously high prevalence and economic burden of heart failure remains a grand challenge to the global health. N-terminal proBNP (NT-proBNP) is an important clinical biomarker for monitoring disease state of heart failure. However, current commercial and research NT-proBNP assays seldom satisfy the clinical requirements. Accordingly, in some aspects, the present disclosure provides a digital counting-based microfluidic nanobiosensor that can quantify NT-proBNP concentration in whole blood samples with a point-of-care (POC) compatible protocol. In some embodiments, the integrated microfluidic device includes a control circuit that automatically runs the digital NT-proBNP immunoassay. In some embodiments, an NT-proBNP-containing whole blood sample mixed with biotinylated detection antibody is passed through a plasma separator and reacts with a capture antibody-functionalized sensor surface. In some embodiments, streptavidin coated gold nanoparticles (GNPs) are then released to mark the surface-bound single NT-proBNP immunocomplexes and recorded with a bright field microscopy image. In some embodiments, the number of captured GNPs is quantified by a digital counting algorithm and correlated to the NT-proBNP concentration in the sample, for example, via a calibration curve. In some embodiments, the assays and devices disclosed herein achieve NT-proBNP detection in the range of about 50-10,000 pg/mL from only about 7 μL of a whole blood sample in about 10 minutes or less. These and other attributes will be apparent upon a complete review of this specification, including the accompanying figures.

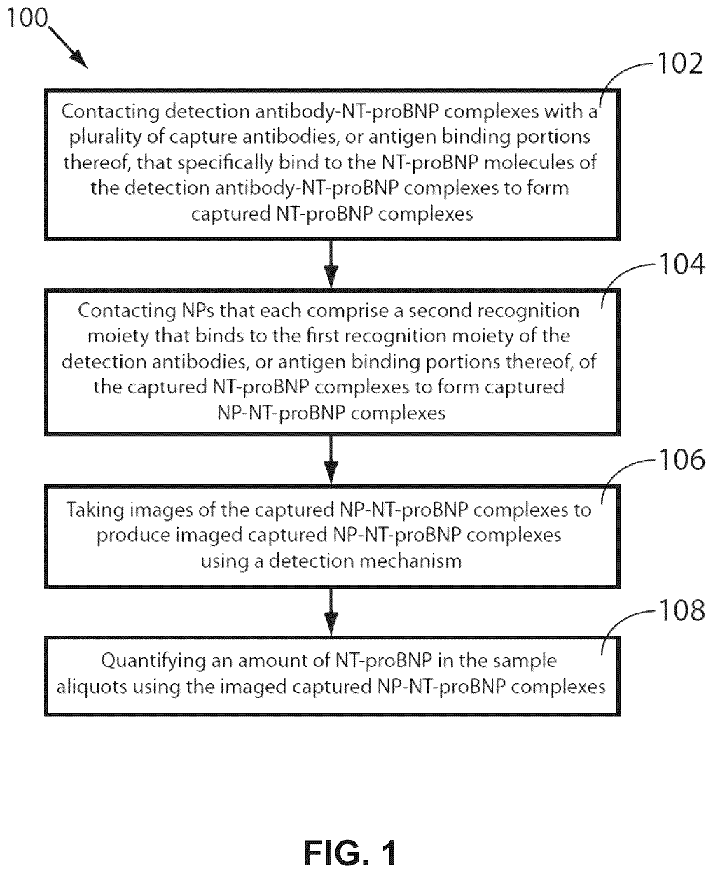

To illustrate, FIG. 1 is a flow chart that schematically shows exemplary method steps quantifying an amount of N-terminal prohormone B-type natriuretic peptide (NT-proBNP) in a whole blood sample according to some aspects disclosed herein. As shown, method 100 includes contacting detection antibody-NT-proBNP complexes with a plurality of capture antibodies, or antigen binding portions thereof, that specifically bind to the NT-proBNP molecules of the detection antibody-NT-proBNP complexes to form captured NT-proBNP complexes (step 102). Typically, the detection antibody-NT-proBNP complexes were formed by contacting a substantially unprocessed NT-proBNP molecule-containing whole blood sample aliquot with a plurality of detection antibodies, or antigen binding portions thereof, that each comprise a first recognition moiety, such as biotin or the like. The detection antibodies, or antigen binding portions thereof, specifically bind to the NT-proBNP molecules in the substantially unprocessed NT-proBNP molecule-containing whole blood sample aliquot to form the detection antibody-NT-proBNP complexes. Method 100 includes contacting nanoparticles (NPs), such as AuNPs that each comprise a second recognition moiety (e.g., streptavidin or the like) that binds to the first recognition moiety of the detection antibodies, or antigen binding portions thereof, of the captured NT-proBNP complexes to form captured NP-NT-proBNP complexes (step 104). As additionally shown, method 100 also includes taking images of the captured NP-NT-proBNP complexes to produce imaged captured NP-NT-proBNP complexes using a detection mechanism (step 106) and quantifying an amount of NT-proBNP in the sample aliquots using the imaged captured NP-NT-proBNP complexes to thereby quantify the amount of NT-proBNP in the whole blood sample (step 108).

In some embodiments, the detection antibodies, or antigen binding portions thereof, specifically bind to a first epitope of the NT-proBNP molecules in which the capture antibodies, or antigen binding portions thereof, specifically bind to a first epitope of the NT-proBNP molecules, and in which the first and second epitopes differ from one another. Typically, the captured NP-NT-proBNP complexes each comprise a single bound NP. In some embodiments, the quantifying step comprises digitally counting the imaged captured NP-NT-proBNP complexes in the images to quantify the amount of NT-proBNP in the whole blood sample. In some embodiments, the quantifying step comprises determining a concentration of the NT-proBNP in the whole blood sample. In some embodiments, the first recognition moiety and corresponding second recognition moiety are independently selected from compounds, such as biotin, streptavidin, avidin, antibodies, antigens, aptamers, proteins, peptides, carbohydrates, or the like.

In some embodiments, method 100 includes flowing the detection antibody-NT-proBNP complexes through a plasma separator prior to contacting the detection antibody-NT-proBNP complexes with the plurality of capture antibodies, or antigen binding portions thereof. In some embodiments, the plurality of capture antibodies, or antigen binding portions thereof, are disposed on a surface of a solid support. In some embodiments, method 100 includes performing at least a portion of the method in a microfluidic digital nanobiosensor (mDNB) device or system. MDNB devices and systems are described further herein. In some embodiments, method 100 includes obtaining the whole blood sample from a subject. In some embodiments, method 100 includes administering, or discontinuing administering, therapy to the subject based at least in part on the amount of NT-proBNP in the whole blood sample obtained from the subject. In some embodiments, method 100 includes generating a therapy recommendation for the subject based at least in part on the amount of NT-proBNP in the whole blood sample obtained from the subject.

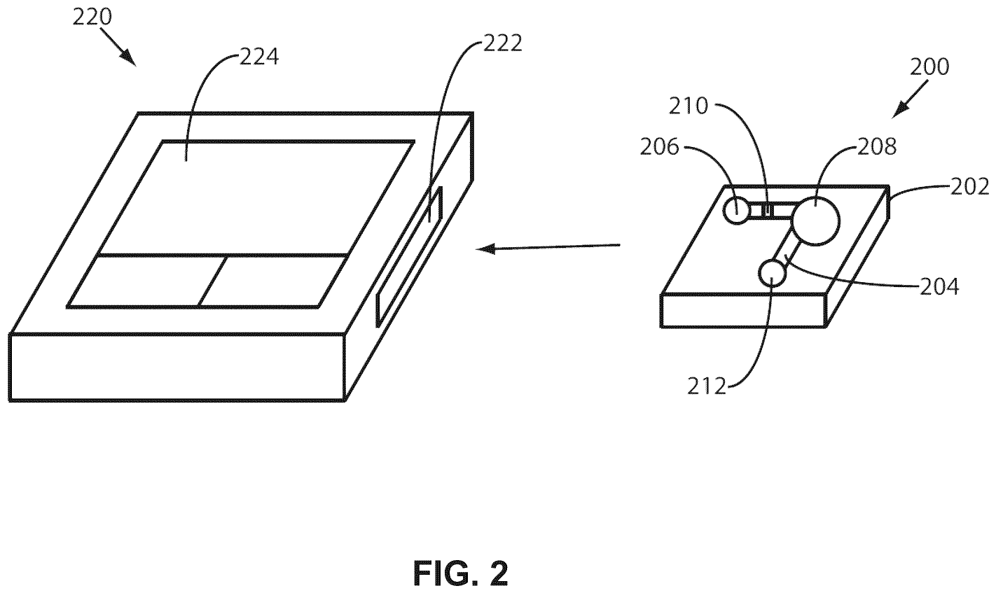

To further illustrate, FIG. 2 is a schematic diagram of an exemplary point-of-care device 220 and microfluidic digital nanobiosensor (mDNB) device 200 (shown as, a microfluidic chip or cartridge) suitable for use with certain aspects disclosed herein. As shown, mDNB device 200 includes body structure 202 that includes microfluidic channel 204 disposed at least partially in body structure 202. MDNB device 200 also includes sample inlet area 206 (shown as including a sample inlet port) disposed at least partially in body structure 202 and in fluid communication with microfluidic channel 204. Sample inlet area 206 is configured to receive sample aliquots that include mixtures of substantially unprocessed N-terminal prohormone B-type natriuretic peptide (NT-proBNP) molecule-containing whole blood and a plurality of detection antibodies, or antigen binding portions thereof, that each comprise a first recognition moiety, which detection antibodies, or antigen binding portions thereof, specifically bind to the NT-proBNP molecules in the whole blood to form detection antibody-NT-proBNP complexes. As shown, mDNB device 200 also includes assay area 208 disposed at least partially in body structure 202 and in fluid communication with microfluidic channel 204. MDNB device 200 also includes plasma separator 210 disposed in a microfluidic channel between sample inlet area 206 and assay area 208.

As described herein, a plurality of capture antibodies, or antigen binding portions thereof, is typically disposed on a surface of assay area 208 in which the capture antibodies, or antigen binding portions thereof, specifically bind to the NT-proBNP molecules of the detection antibody-NT-proBNP complexes when the detection antibody-NT-proBNP complexes are conveyed from sample inlet area 206 to assay area 208 through at least a portion of a microfluidic channel through plasma separator 210 into contact with the plurality of capture antibodies, or antigen binding portions thereof, to form captured NT-proBNP complexes. As additionally shown, mDNB device 200 also includes nanoparticle (NP) reservoir 212 disposed at least partially in body structure 202 and in fluid communication with microfluidic channel 204. NP reservoir 212 is configured to contain NPs that each comprise a second recognition moiety that binds to the first recognition moiety of the detection antibodies, or antigen binding portions thereof, of the captured NT-proBNP complexes when the NPs are conveyed from NP reservoir 212 to assay area 208 through at least a portion of microfluidic channel 204 into contact with the captured NT-proBNP complexes to form captured NP-NT-proBNP complexes.

As additionally shown in FIG. 2, point-of-care (POC) device 220 is configured to receive mDNB device 200 in mDNB device receiving area 222. POC device 220 is also shown as including display screen 224 for inputting instructions and viewing assay results. Although not within view, mDNB device 200 is configured to operably connect to a fluid conveyance mechanism disposed in POC device 220 that effects fluid conveyance through the microfluidic channels of mDNB device 200 to and/or from sample inlet area 206, assay area 208, and NP reservoir 212. Although additionally not within view, mDNB device 200 is also configured to operably interface with a detection mechanism (e.g., a camera, a microscope, or other imaging device) disposed in POC device 220 that images the captured NP-NT-proBNP complexes in assay area 208 to produce imaged captured NP-NT-proBNP complexes such that a controller that is also disposed in POC device 220 and operably connected to the detection mechanism quantifies an amount of NT-proBNP in the sample aliquots from the imaged captured NP-NT-proBNP complexes. Typically, the controller comprises a processor, and a memory communicatively coupled to the processor, the memory storing non-transitory computer executable instructions which, when executed on the processor, perform operations that include digitally counting the imaged captured NP-NT-proBNP complexes in the images to quantify the amount of NT-proBNP in the sample aliquots.

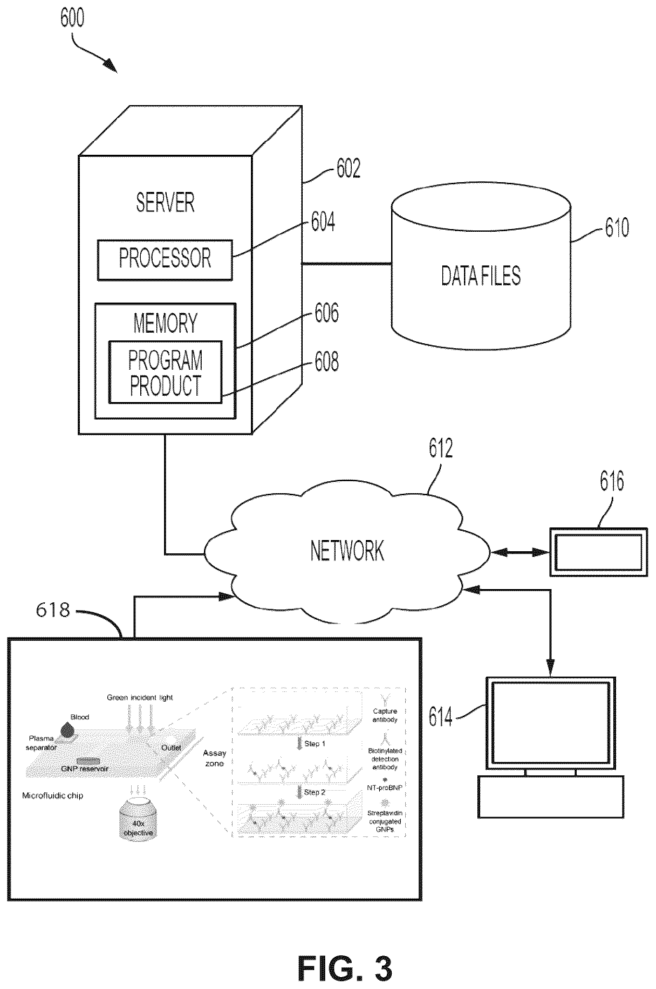

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 3 provides a schematic diagram of an exemplary system suitable for use with implementing at least aspects of the methods disclosed in this application. As shown, system 600 includes at least one controller or computer, e.g., server 602 (e.g., a search engine server), which includes processor 604 and memory, storage device, or memory component 606, and one or more other communication devices 614, 616, (e.g., client-side computer terminals, telephones, tablets, laptops, other mobile devices, etc. (e.g., for receiving imaging data sets or results, etc.) in communication with the remote server 602, through electronic communication network 612, such as the Internet or other internetwork. Communication devices 614, 616 typically include an electronic display (e.g., an internet enabled computer or the like) in communication with, e.g., server 602 computer over network 612 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 600 also includes program product 608 (e.g., for quantifying amounts of NT-proBNP in sample aliquots using imaged captured NP-NT-proBNP complexes as described herein) stored on a computer or machine readable medium, such as, for example, one or more of various types of memory, such as memory 606 of server 602, that is readable by the server 602, to facilitate, for example, a guided search application or other executable by one or more other communication devices, such as 614 (schematically shown as a desktop or personal computer). In some aspects, system 600 optionally also includes at least one database server, such as, for example, server 610 associated with an online website having data stored thereon (e.g., entries patient data sets, etc.) searchable either directly or through search engine server 602. System 600 optionally also includes one or more other servers positioned remotely from server 602, each of which are optionally associated with one or more database servers 610 located remotely or located local to each of the other servers. The other servers can beneficially provide service to geographically remote users and enhance geographically distributed operations.

As understood by those of ordinary skill in the art, memory 606 of the server 602 optionally includes volatile and/or nonvolatile memory including, for example, RAM, ROM, and magnetic or optical disks, among others. It is also understood by those of ordinary skill in the art that although illustrated as a single server, the illustrated configuration of server 602 is given only by way of example and that other types of servers or computers configured according to various other methodologies or architectures can also be used. Server 602 shown schematically in FIG. 3, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 600. As also understood by those of ordinary skill in the art, other user communication devices 614, 616 in these aspects, for example, can be a laptop, desktop, tablet, personal digital assistant (PDA), cell phone, server, or other types of computers. As known and understood by those of ordinary skill in the art, network 612 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 608 is optionally in the form of microcode, programs, cloud computing format, routines, and/or symbolic languages that provide one or more sets of ordered operations that control the functioning of the hardware and direct its operation. Program product 608, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 608 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 608 is optionally copied from the computer-readable medium to a hard disk or a similar intermediate storage medium. When program product 608, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 608 includes non-transitory computer-executable instructions which, when executed by electronic processor 604, perform at least: conveying fluid through a microfluidic channel of an mDNB device as described herein to and/or from a sample inlet area, a assay area, and a NP reservoir using a fluid conveyance mechanism; taking images of captured NP-NT-proBNP complexes in an assay area to produce imaged captured NP-NT-proBNP complexes using a detection mechanism; and quantifying an amount of NT-proBNP in sample aliquots using imaged captured NP-NT-proBNP complexes.

Typically, imaging is obtained using mDNB device or subassembly 218. As shown, mDNB device or subassembly 218 includes a fluid conveyance mechanism for flowing samples and other reagents through microfluidic channels, as described herein. Captured NP-NT-proBNP complexes are counted using the optical imaging system shown (e.g., a bright-field microscope), which includes an objective lens and a CCD camera.

### Example: Microfluidic Digital Nanobiosensor for Point-of-Care Detection of NT-proBNP from Whole Blood

### Introduction

In the present example, we report a microfluidic digital immunoassay (MDIA) for POC compatible detection of NT-proBNP from 7 μL of whole blood. The detection modality is digital counting, which has shown capability of high-sensitivity biomarker detection with high precision. Unlike the ensemble detection methodologies adopted by previously reported sensors, digital counting marks each formed immunocomplex with single gold nanoparticles (GNPs) for direct quantification. As the number of the formed immunocomplexes is the most accurate signal of immunoreaction, digital counting eliminates the noise and interference brought by extra signal transduction and amplification steps, allowing for a lower LoD. Microfluidic biosensors have shown suitability for POC implementation due to their compact size, low sample volume requirement, and high reaction efficiency. When integrated with an on-chip blood processing unit, these microfluidic biosensors further simplify the assay by avoiding manual sample preparation to increase the potential utility in outpatient and home settings. The MDIA includes a microfluidic chip and an automated controller. The chip is integrated with a filter-based plasma separator, a capture antibody prefunctionalized assay zone, and an on-chip GNP reservoir for optical labeling of captured NTproBNP. The surface-bound GNPs are quantified via digital counting from bright-field images and converted to the NTproBNP concentration with a calibration curve. As analyte concentrations increase, optical crowding of the GNPs reduces the digital counting precision. Thus, when the GNP counts are higher than a predetermined threshold, the calibration curve switches to an analog mode based on GNP pixel occupancy. We evaluated the analytical performance of MDIA in undiluted human plasma and whole blood spiked samples. Our results meet the sensitivity, precision, reproducibility, and concentration range requirements for clinical testing with a 10 min assay time. Cross validation of our method was conducted on 15 clinical samples with Roche's Elecsys proBNP II assay. We anticipate that MDIA could become a POC device for decentralized detection of NT-proBNP to assist heart failure patient management.

### Materials and Methods

Materials. 11-Mercaptoundecanoic acid (catalog no. 450561), N-hydroxysuccinimide (NHS, catalog no. 130672), N-(3-dimethylaminopropyl)-N-ethylcarbodiimide hydrochloride (EDC, catalog no. 03450), recombinant streptavidin (catalog no. S4762), Amicon Ultra-0.5 mL centrifugal filters and Tween 20 were purchased from Sigma-Aldrich (St. Louis, MO). EZ-Link NHS-biotin (catalog no. 20217), 1-Step ultra TMB-ELISA substrate solutions (catalog no. 34028), and Zeba spin desalting columns, 7K MWCO, 0.5 mL (catalog no. 89882), and horse serum (catalog no. 26050) were purchased from ThermoFisher Scientific. 1×phosphate buffered saline (PBS, catalog no. 21-040-CV) was purchased from VWR International. Capture antibody (cAb, catalog no. BRJNBNPS108), detection antibody (dAb, catalog no. BRJNBNPS102), and recombinant antigen (catalog no. GRCBNPS101) for human NT-proBNP were purchased from Fapon International Limited. Skim milk powder (catalog no. 02902887) was purchased from MP Biochemical. Reagent diluent (catalog no. DY995), streptavidin conjugated horseradish peroxidase (streptavidin-HRP, catalog no. DY998), stop solution (catalog no. DY994), and high-binding microplate (catalog no. DY990) were purchased from R&D Systems. 40 nm BioReady GNPs (catalog no. AUXR40-5M) were purchased from NanoComposix. Gender unspecified pooled K2EDTA plasma and gender unspecified pooled K2EDTA whole blood from healthy human donors were purchased from BiolVT. Horse serum (catalog no. 26050) was purchased from ThermoFisher Scientific. Vivid plasma separation membrane (catalog no. T9EXPPA0200S00R) was purchased from Pall Life Sciences. Photoresist SU-8 2025 was purchased from MicroChem. Sylgard 184 clear silicone elastomer kit (catalog no. DC4019862) was purchased from Krayden Inc. Silicon wafers were purchased from Avago Technologies. Deidentified serum samples from patients were provided by Mayo Clinic Arizona, under a protocol approved by the Mayo Clinic Institutional Review Board and Biospecimens Subcommittee (IRB No. 19-002558/Bio00017399).

Microfluidic Chip Fabrication. Polydimethylsiloxane (PDMS) soft lithography was used to fabricate the microfluidic chip. A master wafer mold was first fabricated by using photolithography. As shown in FIG. S2, a layer of 35 μm photoresist SU-8 2025 was spin-coated onto a silicon wafer, followed by ultraviolet exposure and chemical development. Then, a 10:1 weight ratio of Sylgard 184 elastomer and curing agent was mixed and cast onto the silicon mold, followed by curing at 60° C. for 2 h. Once the sample was cured, the PDMS was cut, and access holes were punched. The microfluidic chip was assembled by attaching the PDMS stamp onto the functionalized gold surface. The microfluidic channel was then blocked with a blocking buffer for 10 min. Lastly, a GNP reservoir and a plasma separator were plugged onto the chip prior to use.

Reagent Preparation. The calibration curve for MDIA was generated using standard solutions prepared by diluting a stock solution of recombinant NT-proBNP (1 mg/mL) with pooled human plasma to reach spiked-in concentrations of 50, 100, 200, 500, 2000, 5000, and 10 000 pg/mL. Clinical validation was done using residual patient serum samples obtained from the clinical laboratory at Mayo Clinic Arizona. These serum samples were deidentified and transported on ice to our laboratory and stored at 2-8° C. for no more than 6 days. Whole blood spike recovery experiments were performed using recombinant NT-proBNP spiked into human whole blood to reach spiked-in plasma concentrations of 92, 272, and 488 pg/mL.

Microfluidic Flow Controller. The custom-designed compact microfluidic flow controller was developed by using an Arduino Uno R3 board. It consists of 5 solenoid drivers for 2 two-position three-way switch valves (catalog no. 1528-4663-ND, DigiKey), 1 solenoid push valve (catalog no. 1568-1592-ND, DigiKey), 1 needle valve (constructed by gluing a needle tip to a solenoid push valve), and 1 vacuum pump (catalog no. 1528-4700-ND, DigiKey). The 2 switch valves work together to control the connection between the vacuum pump and the microfluidic channel. All valves are connected to routine digital output pins on the Arduino board while the vacuum pump is controlled by a pin capable of pulse width modulation (PWM). A pressure sensor is connected to an analog input pin of the Arduino board to transmit measurements of vacuum pressure supplied to the microfluidic channel in real time. Negative pressure is controlled through proportional-integral-derivative (PID) controlling of the vacuum pump with the pressure sensor signal as feedback. An in-house sketch responsible for executing the control instructions was written using an Arduino Integrated Development Environment.

Microfluidics Digital Immunoassay Operation. For whole blood assays, 7 μL of whole blood sample was mixed with 7 μL of biotinylated dAb solution and loaded into the plasma separator. For plasma and serum assays, 5 μL of sample was mixed with 9 μL of biotinylated dAb to keep a 1:1.8 ratio of plasma/serum to biotinylated dAb as plasma accounts for ˜55% of the whole blood volume. After sample loading, the microcontroller algorithm was initiated to automatically operate the microfluidics: (1) The micro vacuum pump connected to the outlet of the chip applied −5000 Pa pressure to the fluidic channel for 1 min to extract plasma through the membrane filter in the plasma separator into a soft capillary tubing; (2) the plasma separator was pulled off after the extraction, leaving the capillary tubing containing the extracted plasma on the inlet; (3) the plasma sample was driven by a vacuum pressure of −1000 Pa to flow through the assay zone in 5 min; (4) the vacuum pump was turned off and the switch valve actuated to connect the channel to atmosphere to release the residual pressure after the sample completely flowed through; (5) the solenoid push valve was actuated on to disconnect the inlet; (6) the solenoid needle valve was actuated on and off to unseal the GNP reservoir; (7) the vacuum pump was turned on and reconnected via the switch valve to flow the GNP solution into the assay zone with a pressure of −5000 Pa; (8) the pump was turned off and the switch valve was actuated to connect the channel to atmosphere to release the residual pressure for 5 min static incubation of streptavidin-GNPs in the channel. This procedure allows immunocomplex formation of NT-proBNP sandwiched by the surface bound capture antibodies and biotinylated detection antibodies, which is then labeled by the streptavidin-GNPs.

Image Acquisition. Bright-field imaging of the sensor surface with GNP-labeled immunocomplexes was carried out on an Olympus IX-81 optical microscope equipped with an Olympus UPIanFLN 40×/0.75NA objective and a complementary metal-oxide semiconductor (CMOS) scientific camera (Hamamatsu, C11440). Bright-field images of surface-bound GNPs were captured with an exposure time of 1 ms and a 200-frame in-camera rolling average under green light illumination (white light conditioned with an Olympus IF550 green filter). The pixel size of the camera is 6.5 μm, and the image size is 1024×1024 pixels, giving a sensing area of 166×166 μm².

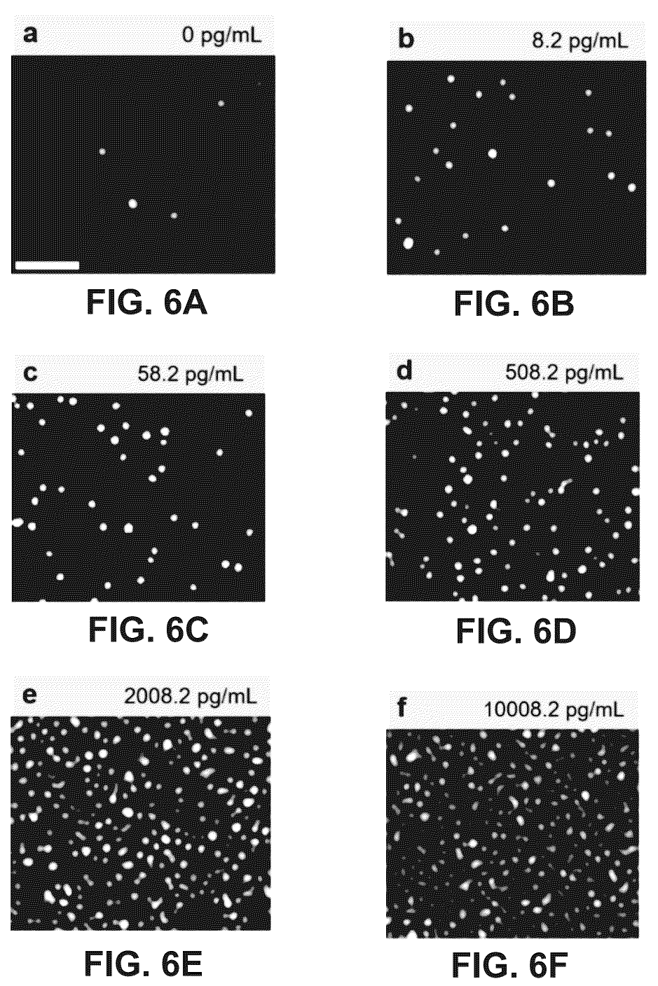

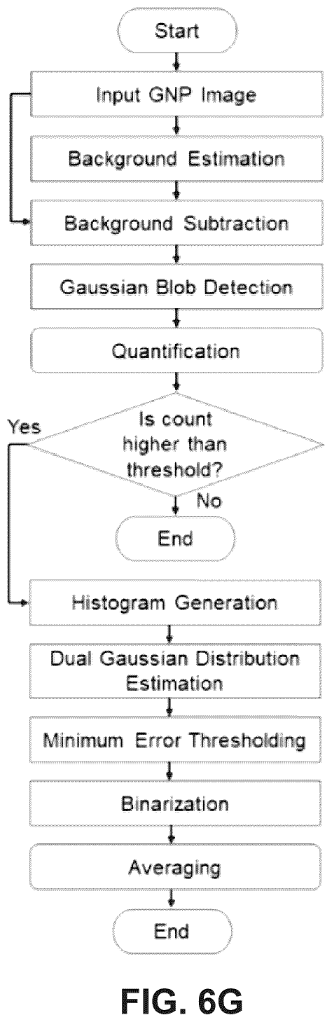

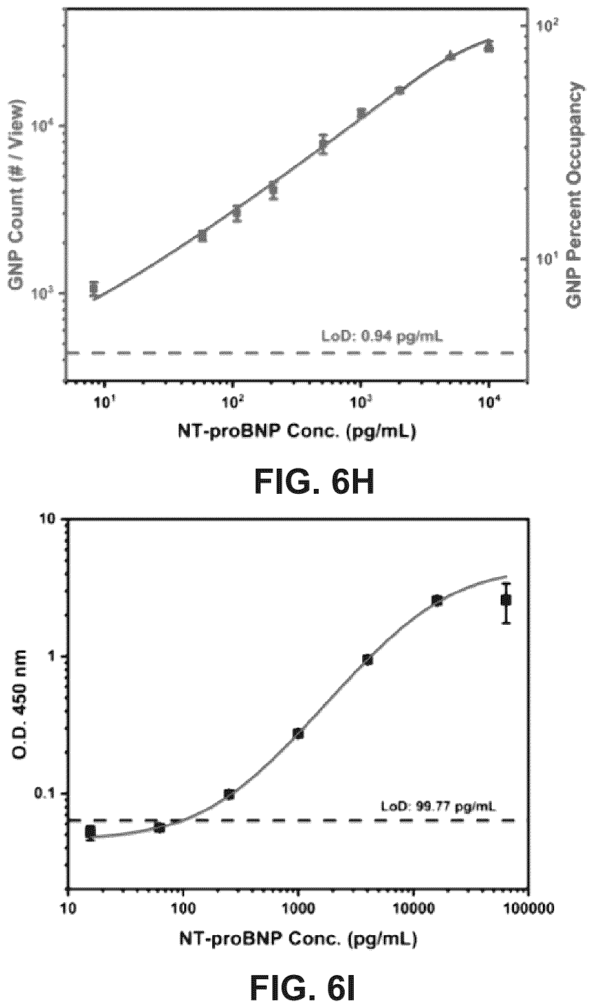

Image Processing. Image processing was carried out using ImageJ. A flowchart of the image processing protocol is illustrated in FIG. 6g. A digital counting algorithm was used for precise quantification of NT-proBNP concentrations lower than 2000 pg/mL. For concentrations higher than 2000 pg/mL, optical viewing of GNPs becomes too crowded to be singly resolved, and thus an image segmentation algorithm (analog mode) based on automatic global histogram thresholding was used to calculate the GNP occupied area. The data processing algorithm goes as follows: (1) A median filter was applied on the raw image to generate a background image estimate; (2) a background-free image was then obtained by subtracting the raw image from the generated background image; (3) Laplacian of Gaussian blob detection was used to count the number of particles on the background-subtracted image using Trackmate; (4) if digital count exceeds the predetermined threshold of 16 000, minimum error thresholding is done to segment the GNP occupied area on the image and calculate GNP pixel occupancy. The minimum error thresholding method models the pixel intensity histograms of the GNPs and the background area as 2 independent normal distributions overlapping at an intensity threshold, T. It then seeks to minimize an error function J(T) calculated from the histogram of the image of interest, which is an estimate of the overlap between the models of the GNPs and the background area. Due to camera pixel noise, some pixels not taken up by the GNPs would have an intensity higher than the thresholding cutoff value, manifesting as noise dots of single pixels on the thresholded image. Therefore, the “analyze particles” command in ImageJ was used to remove such noise dots with a size exclusion value of 3 pixels after thresholding. Finally, the mean intensity of the resultant threshold mask was calculated as the readout signal.

### Results and Discussion

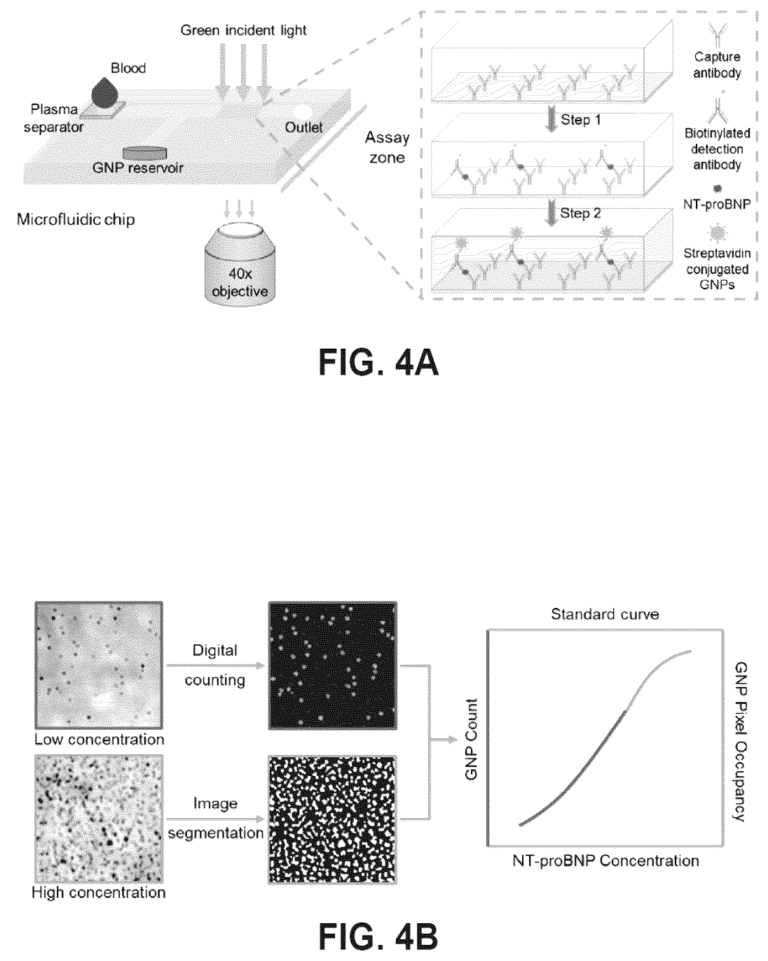

Working Principle of MDIA. FIG. 4 summarizes the principle of MDIA for the NT-proBNP detection. A microfluidic chip serves as the platform for conducting immunoreaction of NT-proBNP. Capture antibody specific to NT-proBNP is preimmobilized to the assay zone on the bottom surface of the microfluidic channel. Surface immobilization of cAb was confirmed using surface plasmon resonance. A gold-coated glass surface was used as thiol-gold chemistry proved to be more reliable and reproducible than glass chemistry for protein conjugation. The microfluidic chip contains a T-shaped channel, which enables the implementation of a 2-step sandwich immunoassay. The 2-step assay permits the formation of NT-proBNP sandwiched by the cAb and dAb at the assay zone and the labeling of such immunocomplexes by individual streptavidin-GNPs. Combining samples with the dAb allows the immunoreaction to occur simultaneously both at the surface and in the solution, which greatly increases the binding efficiency while reducing the assay time and steps.

Labeling of the NT-proBNP immunocomplexes with GNPs allows for discrete quantification via simple optical imaging. GNPs near the size of IgG antibodies can be clearly seen with bright-field imaging due to localized SPR. 40 nm GNP was chosen for optimal balance between ease of detection and binding performance. Since the absorption peak of 40 nm GNPs lies in the green light spectrum, a green incident light centered at 550 nm is used to gain a higher signal-to-noise ratio of GNPs in bright-field imaging.

As shown in FIG. 4B, GNPs appear as dark spots in brightfield images. As the number of GNPs is correlated to the concentration of NT-proBNP in the sample, digital counting is used to calibrate the dose response of NT-proBNP. As the NTproBNP concentration increases, nanoparticles begin to crowd closer than their diffraction limit and cannot be singly resolved. In this case, an analog mode signal processing algorithm is adopted to calculate the GNP pixel occupancy. After minimum error thresholding and noise removal is applied to the raw image data, a thresholded mask is obtained with the pixel intensity of background area set to 0 and that of GNP occupied area set to 255. The GNP pixel occupancy percentage, Oₚᵢₓₑₗ, can be calculated from the mean intensity of the mask as

$${O}_{pixel} = {I}_{total} / {N}_{pixel}$$

where Iₜₒₜₐₗ is the total intensity of the mask and Nₚᵢₓₑₗ is the number of pixels in the mask.

To create a wide dynamic range calibration curve, image data are analyzed using both digital counting and analog methods. As GNPs begin to crowd, the digital count begins to plateau. Thus, an optimal transition point, where the digital curve starts to plateau, has been selected for switching from digital counting to the analog mode. To use the dual-mode standard curve for measuring unknown samples, the digital counting mode is applied first. If signal count exceeds the predetermined threshold, the analog mode is used instead. The combination of digital counting mode and analogue mode enables simultaneous high sensitivity and extended dynamic range.

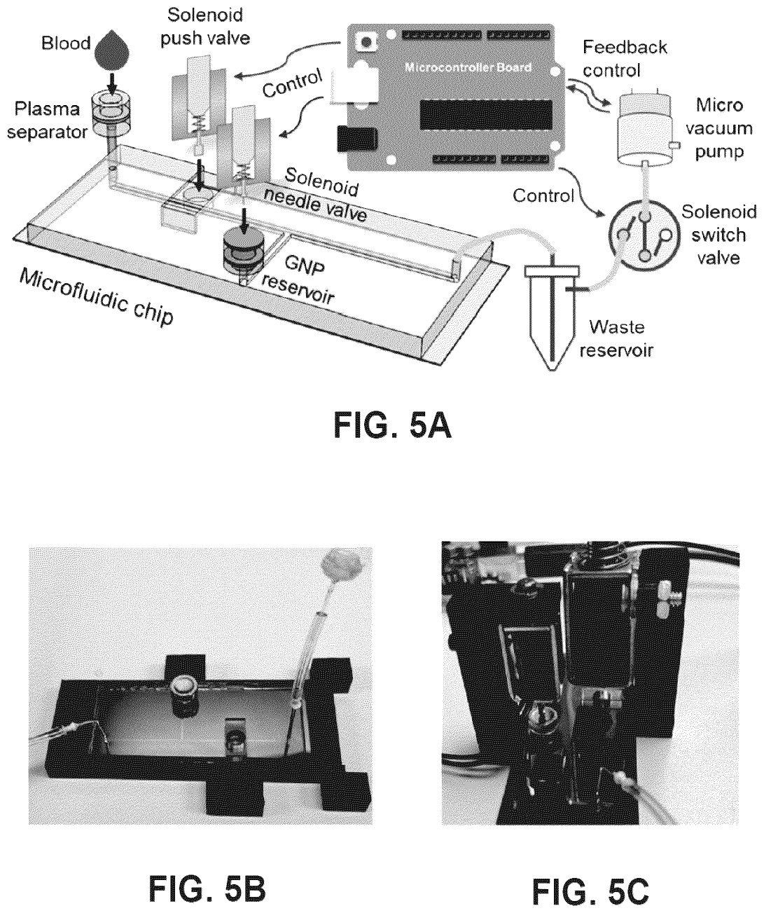

Design of the POC Microfluidic Device. The design of the POC microfluidic device for whole blood-based detection of NT-proBNP is illustrated in FIG. 5. The microfluidic device consists of a microfluidic chip, a micro vacuum pump, and an in-house built fluid control circuit. The microfluidic chip is assembled by first attaching a PDMS slab containing a T-shaped channel to a gold coated glass coverslip through natural adhesion, which can hold the channel in place with fluid flow driven by vacuum withdrawal. Then, a reservoir pouch encapsulating the GNP solution and a plasma membrane filter module are installed in their respective positions at the access hole of the vertical branch and inlet of the horizontal branch of the T channel. FIG. 5b displays a photograph of the assembled microfluidic chip. The micro vacuum pump is connected to the outlet of the microfluidic chip through a waste reservoir to prevent sample contamination while maintaining negative pressure required for driving fluid flow. The in-house built control circuit is used to control the vacuum pump and 3 solenoid valves. As shown in FIG. 5a, a solenoid push valve is placed above the stop valve of the microfluidic chip, which closes the stop valve and disrupts the connection between the inlet and the outlet when it is actuated. Above the GNP reservoir is a solenoid needle valve that punctures the reservoir to release the GNP solution.

The microfluidic chip is for single use and disposable to avoid cross contamination between samples, while the vacuum pump and the fluid control circuit and actuators are reusable as a portable device. While the current design utilizes a single channel with a single biomolecule capture region, it has left room for the addition of multiple channels and independent capture regions for multiplexed biomolecule measurements on a single chip. Overall, our microfluidic device should meet the requirements for POC use when coupled with a portable imaging device.

Detection of NT-proBNP spiked in human plasma. To evaluate the analytical performance of MDIA, we generated a calibration curve using NT-proBNP-spiked human plasma. Recombinant NT-proBNP was spiked into human plasma, forming a series of standard solutions with concentrations covering much of the clinically relevant range of NT-proBNP. The endogenous level of NT-proBNP in the human plasma was determined to be 8.24 pg/mL using conventional ELISA calibrated with NT-proBNP-spiked horse serum samples. This concentration was added to the spiked-in concentrations for MDIA calibration curve fitting. Horse serum was used as the blank control to measure the background signal of MDIA. These spiked standard solutions were loaded to MDIA following the testing protocol described in the methods section, and the corresponding bright-field images for different concentrations were recorded and analyzed. Representative images for horse serum, nonspiked human plasma, and spiked human plasma with spiked-in concentrations of 50, 500, 2000, and 10 000 pg/mL are shown respectively in FIG. 6a-f, showing a clear trend that the number of surface-bound GNPs increases with the concentration of NT-proBNP spiked into the plasma. The background count of GNPs in the horse serum is 248±18, which is primarily caused by nonspecific binding between the GNPs and the sensor surface.

The present background level is significantly lower (p=0.01, Student's t test) than the count of the endogenous NT-proBNP level in the human plasma (1065±98) and thus is adequate for NT-proBNP detection at clinically relevant concentration ranges. Control experiments with no capture antibody immobilized and sensor surface directly blocked with skim milk were conducted, where sensor signal for 1000 pg/mL NT-proBNP was significantly smaller (p=0.01, Student's t test) than that of capture antibody functionalized chips, which supports successful attachment of GNPs through specific antigen-antibody binding.

As illustrated in FIG. 6g, for low concentration samples, each GNP can be resolved as an individual bright spot on the images, whereas for high concentration samples, GNPs overlap and form clusters. We analyzed all the images using both the digital counting and the analog mode image processing and selected the optimal transition point (2000 pg/mL) for switching from digital counting to analog mode. Using this protocol, a hybrid calibration curve was created, as depicted in FIG. 6h. We determined the limit of blank (LoB) and LoD in reference to the Clinical and Laboratory Standards Institute (CLSI) guidelines EP17.27 We can find the signal levels corresponding to LoB and LoD (LoBsignal and LoDsignal) using equations

$${LoB}_{signal} = {Mean}_{blank} + 1.645 * {SD}_{blank} \\ {LoD}_{signal} = LoB + 1.645 * {SD}_{low level sample}$$

where Mean_{blank} and SD_{blank} are the mean and standard deviation of measured blank control signal, which in our case are 248 and 18, respectively. SD_{low level sample} is the standard deviation of the measured low level sample signal, which in our case is 98 for the 8.24 pg/mL sample. Following these equations, the LoB was back calculated from the calibration curve to be 0.03 pg/mL and the LoD was 0.94 pg/mL, outperforming the sensitivity needs for clinical NT-proBNP testing. The upper limit of detection is 10 000 pg/mL for the MDIA, above which the output signal plateaus due to depletion of the GNPs, but samples with concentrations beyond 10 000 pg/mL can still be categorized as “very high”. In addition, the maximum percentage coefficient of variation (CV) for the calibration curve is 13% at 500 pg/mL, which demonstrates satisfactory precision of our method as reference to a ≤15% total CV recommendation by the European Society of Cardiology working group on acute cardiac care.

Conventional sandwich ELISA was also performed to compare its analytical performance against MDIA. The same antibody pair, assay diluent, blocking agent, and washing buffer were used. To allow more straightforward comparison, the incubation time of the mixture of samples and biotinylated dAb, as well as the streptavidin-HRP solution, was changed to 5 min. As shown in FIG. 6i, the LoD for the conventional ELISA is 99.77 pg/mL with signal plateauing at 16 000 pg/mL. This shows that MDIA has a LoD 100 times lower than conventional ELISA.

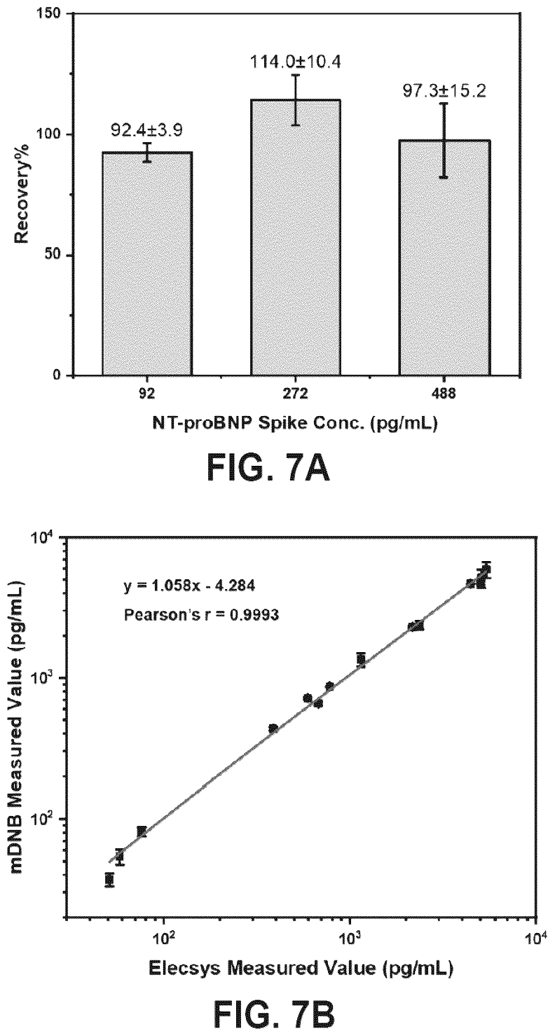

Validation in whole blood spiked samples. To test MDIA for the POC detection of NT-proBNP, we conducted a spike and recovery study. Recombinant human NT-proBNP was spiked into whole blood from healthy human donors to reach nominal spiked-in plasma concentrations of 1000, 500, and 200 pg/mL. However, the actual plasma concentration of NT-proBNP could be biased relative to the nominal concentration due to errors in the dilution process and variation in hematocrit. To confirm the real plasma concentrations, we used conventional ELISA to measure the spiked samples after extracting their plasma components by centrifugation. The actual spiked-in plasma concentrations corresponding to the nominal 1000, 500, and 200 pg/mL were 488, 272, and 92 pg/mL, respectively. These spiked whole blood samples were also measured using MDIA, and their NT-proBNP concentrations were determined using the calibration curve in FIG. 6h. The spiked-in concentrations were then calculated by subtracting the endogenous NT-proBNP level from the MDIA measured concentrations. Based on these data, recoveries were calculated for each of the 3 spiked concentrations. As shown in FIG. 7a, the recovery of MDIA measured concentrations is within 20% of true values. These results suggest that MDIA can reliably measure NTproBNP concentration in whole blood samples and substantiate its POC application when plasma samples are not convenient to obtain via centrifugation.

Evaluation in Clinical Samples. To evaluate clinical application of MDIA, we compared it against the FDA cleared Roche Elecsys proBNP II assay using patient serum samples. The Elecsys proBNP II assay was performed on patient samples using a Roche Cobas Pro instrument at Mayo Clinic Arizona as part of the routine clinical diagnostic protocol for patients suspected of heart failure. Residual patient serum samples were then deidentified and transported to our laboratory to be measured with MDIA. A total of 15 patient samples were tested, and measurement results of our method were plotted against those of the Elecsys proBNP II assay, shown in FIG. 7b. Pearson's correlation analysis was conducted based on these results, which indicates a strong linear correlation between our method and Roche's assay with a slope of 1.073 and a correlation coefficient of 0.998. This correlation analysis suggests that our method agrees well with the Elecsys proBNP II assay and holds promise for clinical use.

Comparison with Existing POC NT-proBNP Assays. Even though commercial BNP and NTproBNP assays can work with whole blood samples, nearly all of them require large quantities of intravenous blood sampling. The sample volume, detection limit, and assay time of the MDIA are the lowest compared to those of all of the commercial assays.

Overall, the MDIA shows strong potential for becoming a POC device for convenient, decentralized monitoring of heart failure patients. Nevertheless, several additional technological integrations and optimizations should be addressed before the MDIA can be put into practical application. First, a portable imaging device must be integrated for on-site signal readout and processing. Second, an in-channel microfluidic mixer should be incorporated to eliminate the need for manual mixing of the blood sample and the dAb. Lastly, a reliable glass surface functionalization method could be implemented to avoid the use of gold surface and minimize the per-unit cost.

### CONCLUSIONS

We developed a microfluidic digital immunoassay that can rapidly detect NT-proBNP in 7 μL of whole blood. Using an in-house built microcontroller-operated microfluidic device with plasma separation, the NT-proBNP immunoreaction can be automatically performed on the microfluidic chip. This enables POC detection and quantification of NT-proBNP directly from whole blood samples. Digital counting was utilized to transduce the binding signal by quantifying the number of single GNP-labeled NT-proBNP immunocomplexes formed on the sensor surface. Combining digital counting with a thresholding-based image segmentation algorithm, we achieved highly sensitive and accurate detection of NT-proBNP with a dynamic range covering much of the clinically relevant concentrations. The LoD of the prototype MDIA device is 0.94 pg/mL with precision within 13% for concentrations up to 10 000 pg/mL, while assay time takes only 10 min. We also evaluated the clinical applicability of the developed biosensor by comparing it to the Elecsys proBNP II assay using 15 patient serum samples. A strong linear correlation (r=0.998) obtained from the comparison supports the potential of our method in clinical use. This evidence leads us to believe that the microfluidic digital immunoassay is a promising POC method for decentralized monitoring of heart failure.

Some further aspects are also defined in the following clauses:

Clause 1: A method of quantifying an amount of N-terminal prohormone B-type natriuretic peptide (NT-proBNP) in a whole blood sample, comprising: contacting detection antibody-NT-proBNP complexes with a plurality of capture antibodies, or antigen binding portions thereof, that specifically bind to the NT-proBNP molecules of the detection antibody-NT-proBNP complexes to form captured NT-proBNP complexes, wherein the detection antibody-NT-proBNP complexes were formed by contacting a substantially unprocessed NT-proBNP molecule-containing whole blood sample aliquot with a plurality of detection antibodies, or antigen binding portions thereof, that each comprise a first recognition moiety, which detection antibodies, or antigen binding portions thereof, specifically bind to the NT-proBNP molecules in the substantially unprocessed NT-proBNP molecule-containing whole blood sample aliquot to form the detection antibody-NT-proBNP complexes; contacting nanoparticles (NPs) that each comprise a second recognition moiety that binds to the first recognition moiety of the detection antibodies, or antigen binding portions thereof, of the captured NT-proBNP complexes to form captured NP-NT-proBNP complexes; taking images of the captured NP-NT-proBNP complexes to produce imaged captured NP-NT-proBNP complexes using a detection mechanism; and, quantifying an amount of NT-proBNP in the sample aliquots using the imaged captured NP-NT-proBNP complexes, thereby quantifying the amount of NT-proBNP in the whole blood sample.

Clause 2: The method of Clause 1, wherein the detection antibodies, or antigen binding portions thereof, specifically bind to a first epitope of the NT-proBNP molecules, wherein the capture antibodies, or antigen binding portions thereof, specifically bind to a first epitope of the NT-proBNP molecules, and wherein the first and second epitopes differ from one another.

Clause 3: The method of Clause 1 or Clause 2, wherein the captured NP-NT-proBNP complexes each comprise a single bound NP.

Clause 4: The method of any one of Clauses 1-3, wherein the quantifying step comprises digitally counting the imaged captured NP-NT-proBNP complexes in the images to quantify the amount of NT-proBNP in the whole blood sample.

Clause 5: The method of any one of Clauses 1-4, wherein the quantifying step comprises determining a concentration of the NT-proBNP in the whole blood sample.

Clause 6: The method of any one of Clauses 1-5, wherein the first recognition moiety is a compound selected from the group consisting of: biotin, streptavidin, avidin, an antibody, an antigen, an aptamer, a protein, a peptide, and a carbohydrate.

Clause 7: The method of any one of Clauses 1-6, wherein the second recognition moiety is a compound selected from the group consisting of: biotin, streptavidin, avidin, an antibody, an antigen, an aptamer, a protein, a peptide, and a carbohydrate.

Clause 8: The method of any one of Clauses 1-7, comprising flowing the detection antibody-NT-proBNP complexes through a plasma separator prior to contacting the detection antibody-NT-proBNP complexes with the plurality of capture antibodies, or antigen binding portions thereof.

Clause 9: The method of any one of Clauses 1-8, wherein the plurality of capture antibodies, or antigen binding portions thereof, are disposed on a surface of a solid support.

Clause 10: The method of any one of Clauses 1-9, comprising performing at least a portion of the method in a microfluidic digital nanobiosensor (mDNB) device or system.

Clause 11: The method of any one of Clauses 1-10, comprising obtaining the whole blood sample from a subject.

Clause 12: The method of any one of Clauses 1-11, comprising administering, or discontinuing administering, therapy to the subject based at least in part on the amount of NT-proBNP in the whole blood sample obtained from the subject.

Clause 13: The method of any one of Clauses 1-12, comprising generating a therapy recommendation for the subject based at least in part on the amount of NT-proBNP in the whole blood sample obtained from the subject.

Clause 14: A microfluidic digital nanobiosensor (mDNB) device, comprising: a body structure comprising at least one microfluidic channel disposed at least partially in the body structure; a sample inlet area disposed at least partially in the body structure and in fluid communication with the microfluidic channel, wherein the sample inlet area is configured to receive sample aliquots that comprise mixtures of substantially unprocessed N-terminal prohormone B-type natriuretic peptide (NT-proBNP) molecule-containing whole blood and a plurality of detection antibodies, or antigen binding portions thereof, that each comprise a first recognition moiety, which detection antibodies, or antigen binding portions thereof, specifically bind to the NT-proBNP molecules in the whole blood to form detection antibody-NT-proBNP complexes; an assay area disposed at least partially in the body structure and in fluid communication with the microfluidic channel; a plasma separator disposed in the microfluidic channel between the sample inlet area and the assay area; a plurality of capture antibodies, or antigen binding portions thereof, disposed on a surface of the assay area, wherein the capture antibodies, or antigen binding portions thereof, specifically bind to the NT-proBNP molecules of the detection antibody-NT-proBNP complexes when the detection antibody-NT-proBNP complexes are conveyed from the sample inlet area to the assay area through at least a portion of the microfluidic channel through the plasma separator into contact with the plurality of capture antibodies, or antigen binding portions thereof, to form captured NT-proBNP complexes; and, a nanoparticle (NP) reservoir disposed at least partially in the body structure and in fluid communication with the microfluidic channel, which NP reservoir is configured to contain NPs that each comprise a second recognition moiety that binds to the first recognition moiety of the detection antibodies, or antigen binding portions thereof, of the captured NT-proBNP complexes when the NPs are conveyed from the NP reservoir to the assay area through at least a portion of the microfluidic channel into contact with the captured NT-proBNP complexes to form captured NP-NT-proBNP complexes; wherein the mDNB device is configured to operably connect to a fluid conveyance mechanism that effects fluid conveyance through the microfluidic channel to and/or from the sample inlet area, the assay area, and the NP reservoir; and wherein the mDNB device is configured to operably interface with a detection mechanism that images the captured NP-NT-proBNP complexes in the assay area to produce imaged captured NP-NT-proBNP complexes such that a controller operably connected to the detection mechanism quantifies an amount of NT-proBNP in the sample aliquots from the imaged captured NP-NT-proBNP complexes.

Clause 15: The mDNB device of Clause 14, wherein the detection antibodies, or antigen binding portions thereof, specifically bind to a first epitope of the NT-proBNP molecules, wherein the capture antibodies, or antigen binding portions thereof, specifically bind to a first epitope of the NT-proBNP molecules, and wherein the first and second epitopes differ from one another.

Clause 16: The mDNB device of Clause 14 or Clause 15, wherein the captured NP-NT-proBNP complexes each comprise a single bound NP.

Clause 17: The mDNB device of any one of Clauses 14-16, wherein the controller comprises a processor, and a memory communicatively coupled to the processor, the memory storing non-transitory computer executable instructions which, when executed on the processor, perform operations comprising: digitally counting the imaged captured NP-NT-proBNP complexes in the images to quantify the amount of NT-proBNP in the sample aliquots.

Clause 18: The mDNB device of any one of Clauses 14-17, wherein the amount of NT-proBNP in the sample aliquots comprises a concentration of the NT-proBNP in the sample aliquots.

Clause 19: The mDNB device of any one of Clauses 14-18, wherein the detection mechanism comprises a bright-field microscope.

Clause 20: The mDNB device of any one of Clauses 14-19, wherein the NPs comprise metallic nanoparticles (MNPs).

Clause 21: A kit comprising the mDNB device of any one of Clauses 14-20.

Clause 22: The mDNB device of any one of Clauses 14-21, wherein the sample inlet area comprises a sample inlet port.

Clause 23: The mDNB device of any one of Clauses 14-22, wherein a microfluidic chip or cartridge comprises the mDNB device.

Clause 24: The mDNB device of any one of Clauses 14-23, wherein a point-of-care device comprises or is configured to receive the mDNB device.

Clause 25: The mDNB device of any one of Clauses 14-24, wherein the first recognition moiety is a compound selected from the group consisting of: biotin, streptavidin, avidin, an antibody, an antigen, an aptamer, a protein, a peptide, and a carbohydrate.

Clause 26: The mDNB device of any one of Clauses 14-25, wherein the second recognition moiety is a compound selected from the group consisting of: biotin, streptavidin, avidin, an antibody, an antigen, an aptamer, a protein, a peptide, and a carbohydrate.

Clause 27: A microfluidic digital nanobiosensor (mDNB) system, comprising: a mDNB device receiving area structured to receive at least one mDNB device that comprises: a body structure comprising at least one microfluidic channel disposed at least partially in the body structure; a sample inlet area disposed at least partially in the body structure and in fluid communication with the microfluidic channel, wherein the sample inlet area is configured to receive sample aliquots that comprise mixtures of substantially unprocessed N-terminal prohormone B-type natriuretic peptide (NT-proBNP) molecule-containing whole blood and a plurality of detection antibodies, or antigen binding portions thereof, that each comprise a first recognition moiety, which detection antibodies, or antigen binding portions thereof, specifically bind to the NT-proBNP molecules in the whole blood to form detection antibody-NT-proBNP complexes; an assay area disposed at least partially in the body structure and in fluid communication with the microfluidic channel; a plasma separator disposed in the microfluidic channel between the sample inlet area and the assay area; a plurality of capture antibodies, or antigen binding portions thereof, disposed on a surface of the assay area, wherein the capture antibodies, or antigen binding portions thereof, specifically bind to the NT-proBNP molecules of the detection antibody-NT-proBNP complexes when the detection antibody-NT-proBNP complexes are conveyed from the sample inlet area to the assay area through at least a portion of the microfluidic channel through the plasma separator into contact with the plurality of capture antibodies, or antigen binding portions thereof, to form captured NT-proBNP complexes; and, a nanoparticle (NP) reservoir disposed at least partially in the body structure and in fluid communication with the microfluidic channel, which NP reservoir is configured to contain NPs that each comprise a second recognition moiety that binds to the first recognition moiety of the detection antibodies, or antigen binding portions thereof, of the captured NT-proBNP complexes when the NPs are conveyed from the NP reservoir to the assay area through at least a portion of the microfluidic channel into contact with the captured NT-proBNP complexes to form captured NP-NT-proBNP complexes; a fluid conveyance mechanism that operably connects to the mDNB device when the mDNB device is received in the mDNB device receiving area, which fluid conveyance mechanism is configured to effect fluid conveyance through the microfluidic channel to and/or from the sample inlet area, the assay area, and the NP reservoir; a detection mechanism that is configured to take images of the captured NP-NT-proBNP complexes in the assay area to produce imaged captured NP-NT-proBNP complexes when the mDNB device is received in the mDNB device receiving area; and, a controller comprises a processor, and a memory communicatively directly or remotely coupled to the processor, the memory storing non-transitory computer executable instructions which, when executed on the processor, perform operations comprising: conveying the fluid through the microfluidic channel to and/or from the sample inlet area, the assay area, and the NP reservoir using the fluid conveyance mechanism; taking the images of the captured NP-NT-proBNP complexes in the assay area to produce the imaged captured NP-NT-proBNP complexes using the detection mechanism; and quantifying an amount of NT-proBNP in the sample aliquots using the imaged captured NP-NT-proBNP complexes.

Clause 28: The mDNB system of Clause 27, wherein the detection antibodies, or antigen binding portions thereof, specifically bind to a first epitope of the NT-proBNP molecules, wherein the capture antibodies, or antigen binding portions thereof, specifically bind to a first epitope of the NT-proBNP molecules, and wherein the first and second epitopes differ from one another.

Clause 29: The mDNB system of Clause 27 or Clause 28, wherein the captured NP-NT-proBNP complexes each comprise a single bound NP.

Clause 30: The mDNB system of any one of Clauses 27-29, wherein the amount of NT-proBNP in the sample aliquots comprises a concentration of the NT-proBNP in the sample aliquots.

Clause 31: The mDNB system of any one of Clauses 27-30, wherein the detection mechanism comprises a bright-field microscope.

Clause 32: The mDNB system of any one of Clauses 27-31, wherein the NPs comprise metallic nanoparticles (MNPs).

Clause 33: The mDNB system of any one of Clauses 27-32, wherein a microfluidic chip or cartridge comprises the mDNB device.

Clause 34: The mDNB system of any one of Clauses 27-33, wherein the system comprises a point-of-care device that comprises or is configured to receive the mDNB device.

Clause 35: The mDNB system of any one of Clauses 27-34, wherein the first recognition moiety is a compound selected from the group consisting of: biotin, streptavidin, avidin, an antibody, an antigen, an aptamer, a protein, a peptide, and a carbohydrate.

Clause 36: The mDNB system of any one of Clauses 27-35, wherein the second recognition moiety is a compound selected from the group consisting of: biotin, streptavidin, avidin, an antibody, an antigen, an aptamer, a protein, a peptide, and a carbohydrate.

Clause 37: The mDNB system of any one of Clauses 27-36, wherein the mDNB system is configured to detect the NT-proBNP molecules in a range of about 1-10,000 pg/mL from less than about 10 μL of a given whole blood sample in about 15 minutes or less.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of quantifying an amount of N-terminal prohormone B-type natriuretic peptide (NT-proBNP) in a whole blood sample, comprising:
contacting detection antibody-NT-proBNP complexes with a plurality of capture antibodies, or antigen binding portions thereof, that specifically bind to the NT-proBNP molecules of the detection antibody-NT-proBNP complexes to form captured NT-proBNP complexes, wherein the detection antibody-NT-proBNP complexes were formed by contacting a substantially unprocessed NT-proBNP molecule-containing whole blood sample aliquot with a plurality of detection antibodies, or antigen binding portions thereof, that each comprise a first recognition moiety, which detection antibodies, or antigen binding portions thereof, specifically bind to the NT-proBNP molecules in the substantially unprocessed NT-proBNP molecule-containing whole blood sample aliquot to form the detection antibody-NT-proBNP complexes;
contacting nanoparticles (NPs) that each comprise a second recognition moiety that binds to the first recognition moiety of the detection antibodies, or antigen binding portions thereof, of the captured NT-proBNP complexes to form captured NP-NT-proBNP complexes;
taking images of the captured NP-NT-proBNP complexes to produce imaged captured NP-NT-proBNP complexes using a detection mechanism; and,
quantifying an amount of NT-proBNP in the sample aliquots using the imaged captured NP-NT-proBNP complexes, thereby quantifying the amount of NT-proBNP in the whole blood sample.

2. The method of claim 1, wherein the detection antibodies, or antigen binding portions thereof, specifically bind to a first epitope of the NT-proBNP molecules, wherein the capture antibodies, or antigen binding portions thereof, specifically bind to a first epitope of the NT-proBNP molecules, and wherein the first and second epitopes differ from one another.

3. The method of claim 1, wherein the captured NP-NT-proBNP complexes each comprise a single bound NP.

4. The method of claim 1, wherein the quantifying step comprises digitally counting the imaged captured NP-NT-proBNP complexes in the images to quantify the amount of NT-proBNP in the whole blood sample.

5. The method of claim 1, wherein the quantifying step comprises determining a concentration of the NT-proBNP in the whole blood sample.

6. The method of claim 1, wherein the first recognition moiety is a compound selected from the group consisting of: biotin, streptavidin, avidin, an antibody, an antigen, an aptamer, a protein, a peptide, and a carbohydrate.

7. The method of claim 1, wherein the second recognition moiety is a compound selected from the group consisting of: biotin, streptavidin, avidin, an antibody, an antigen, an aptamer, a protein, a peptide, and a carbohydrate.

8. The method of claim 1, comprising flowing the detection antibody-NT-proBNP complexes through a plasma separator prior to contacting the detection antibody-NT-proBNP complexes with the plurality of capture antibodies, or antigen binding portions thereof.

9. The method of claim 1, wherein the plurality of capture antibodies, or antigen binding portions thereof, are disposed on a surface of a solid support.

10. The method of claim 1, comprising performing at least a portion of the method in a microfluidic digital nanobiosensor (mDNB) device or system.

11. The method of claim 1, comprising obtaining the whole blood sample from a subject.

12. The method of claim 11, comprising administering, or discontinuing administering, therapy to the subject based at least in part on the amount of NT-proBNP in the whole blood sample obtained from the subject.

13. The method of claim 11, comprising generating a therapy recommendation for the subject based at least in part on the amount of NT-proBNP in the whole blood sample obtained from the subject.

14. A microfluidic digital nanobiosensor (mDNB) device, comprising:
a body structure comprising at least one microfluidic channel disposed at least partially in the body structure;
a sample inlet area disposed at least partially in the body structure and in fluid communication with the microfluidic channel, wherein the sample inlet area is configured to receive sample aliquots that comprise mixtures of substantially unprocessed N-terminal prohormone B-type natriuretic peptide (NT-proBNP) molecule-containing whole blood and a plurality of detection antibodies, or antigen binding portions thereof, that each comprise a first recognition moiety, which detection antibodies, or antigen binding portions thereof, specifically bind to the NT-proBNP molecules in the whole blood to form detection antibody-NT-proBNP complexes;
an assay area disposed at least partially in the body structure and in fluid communication with the microfluidic channel;
a plasma separator disposed in the microfluidic channel between the sample inlet area and the assay area;
a plurality of capture antibodies, or antigen binding portions thereof, disposed on a surface of the assay area, wherein the capture antibodies, or antigen binding portions thereof, specifically bind to the NT-proBNP molecules of the detection antibody-NT-proBNP complexes when the detection antibody-NT-proBNP complexes are conveyed from the sample inlet area to the assay area through at least a portion of the microfluidic channel through the plasma separator into contact with the plurality of capture antibodies, or antigen binding portions thereof, to form captured NT-proBNP complexes; and,
a nanoparticle (NP) reservoir disposed at least partially in the body structure and in fluid communication with the microfluidic channel, which NP reservoir is configured to contain NPs that each comprise a second recognition moiety that binds to the first recognition moiety of the detection antibodies, or antigen binding portions thereof, of the captured NT-proBNP complexes when the NPs are conveyed from the NP reservoir to the assay area through at least a portion of the microfluidic channel into contact with the captured NT-proBNP complexes to form captured NP-NT-proBNP complexes;
wherein the mDNB device is configured to operably connect to a fluid conveyance mechanism that effects fluid conveyance through the microfluidic channel to and/or from the sample inlet area, the assay area, and the NP reservoir; and
wherein the mDNB device is configured to operably interface with a detection mechanism that images the captured NP-NT-proBNP complexes in the assay area to produce imaged captured NP-NT-proBNP complexes such that a controller operably connected to the detection mechanism quantifies an amount of NT-proBNP in the sample aliquots from the imaged captured NP-NT-proBNP complexes.

15. The mDNB device of claim 14, wherein the detection antibodies, or antigen binding portions thereof, specifically bind to a first epitope of the NT-proBNP molecules, wherein the capture antibodies, or antigen binding portions thereof, specifically bind to a first epitope of the NT-proBNP molecules, and wherein the first and second epitopes differ from one another.

16. The mDNB device of claim 14, wherein the captured NP-NT-proBNP complexes each comprise a single bound NP.

17. The mDNB device of claim 14, wherein the controller comprises a processor, and a memory communicatively coupled to the processor, the memory storing non-transitory computer executable instructions which, when executed on the processor, perform operations comprising: digitally counting the imaged captured NP-NT-proBNP complexes in the images to quantify the amount of NT-proBNP in the sample aliquots.

18. The mDNB device of claim 14, wherein the amount of NT-proBNP in the sample aliquots comprises a concentration of the NT-proBNP in the sample aliquots.

19. The mDNB device of claim 14, wherein the detection mechanism comprises a bright-field microscope.

20. The mDNB device of claim 14, wherein the NPs comprise metallic nanoparticles (MNPs).

21. A kit comprising the mDNB device of claim 14.

22. The mDNB device of claim 14, wherein the sample inlet area comprises a sample inlet port.

23. The mDNB device of claim 14, wherein a microfluidic chip or cartridge comprises the mDNB device.

24. The mDNB device of claim 14, wherein a point-of-care device comprises or is configured to receive the mDNB device.

25. The mDNB device of claim 14, wherein the first recognition moiety is a compound selected from the group consisting of: biotin, streptavidin, avidin, an antibody, an antigen, an aptamer, a protein, a peptide, and a carbohydrate.

26. The mDNB device of claim 14, wherein the second recognition moiety is a compound selected from the group consisting of: biotin, streptavidin, avidin, an antibody, an antigen, an aptamer, a protein, a peptide, and a carbohydrate.

27. A microfluidic digital nanobiosensor (mDNB) system, comprising:
a mDNB device receiving area structured to receive at least one mDNB device that comprises:
a body structure comprising at least one microfluidic channel disposed at least partially in the body structure;
a sample inlet area disposed at least partially in the body structure and in fluid communication with the microfluidic channel, wherein the sample inlet area is configured to receive sample aliquots that comprise mixtures of substantially unprocessed N-terminal prohormone B-type natriuretic peptide (NT-proBNP) molecule-containing whole blood and a plurality of detection antibodies, or antigen binding portions thereof, that each comprise a first recognition moiety, which detection antibodies, or antigen binding portions thereof, specifically bind to the NT-proBNP molecules in the whole blood to form detection antibody-NT-proBNP complexes;
an assay area disposed at least partially in the body structure and in fluid communication with the microfluidic channel;
a plasma separator disposed in the microfluidic channel between the sample inlet area and the assay area;
a plurality of capture antibodies, or antigen binding portions thereof, disposed on a surface of the assay area, wherein the capture antibodies, or antigen binding portions thereof, specifically bind to the NT-proBNP molecules of the detection antibody-NT-proBNP complexes when the detection antibody-NT-proBNP complexes are conveyed from the sample inlet area to the assay area through at least a portion of the microfluidic channel through the plasma separator into contact with the plurality of capture antibodies, or antigen binding portions thereof, to form captured NT-proBNP complexes; and,
a nanoparticle (NP) reservoir disposed at least partially in the body structure and in fluid communication with the microfluidic channel, which NP reservoir is configured to contain NPs that each comprise a second recognition moiety that binds to the first recognition moiety of the detection antibodies, or antigen binding portions thereof, of the captured NT-proBNP complexes when the NPs are conveyed from the NP reservoir to the assay area through at least a portion of the microfluidic channel into contact with the captured NT-proBNP complexes to form captured NP-NT-proBNP complexes;
a fluid conveyance mechanism that operably connects to the mDNB device when the mDNB device is received in the mDNB device receiving area, which fluid conveyance mechanism is configured to effect fluid conveyance through the microfluidic channel to and/or from the sample inlet area, the assay area, and the NP reservoir;
a detection mechanism that is configured to take images of the captured NP-NT-proBNP complexes in the assay area to produce imaged captured NP-NT-proBNP complexes when the mDNB device is received in the mDNB device receiving area; and,
a controller comprises a processor, and a memory communicatively directly or remotely coupled to the processor, the memory storing non-transitory computer executable instructions which, when executed on the processor, perform operations comprising:
conveying the fluid through the microfluidic channel to and/or from the sample inlet area, the assay area, and the NP reservoir using the fluid conveyance mechanism;
taking the images of the captured NP-NT-proBNP complexes in the assay area to produce the imaged captured NP-NT-proBNP complexes using the detection mechanism; and
quantifying an amount of NT-proBNP in the sample aliquots using the imaged captured NP-NT-proBNP complexes.

28. The mDNB system of claim 27, wherein the detection antibodies, or antigen binding portions thereof, specifically bind to a first epitope of the NT-proBNP molecules, wherein the capture antibodies, or antigen binding portions thereof, specifically bind to a first epitope of the NT-proBNP molecules, and wherein the first and second epitopes differ from one another.

29. The mDNB system of claim 27, wherein the captured NP-NT-proBNP complexes each comprise a single bound NP.

30. The mDNB system of claim 27, wherein the amount of NT-proBNP in the sample aliquots comprises a concentration of the NT-proBNP in the sample aliquots.

31. The mDNB system of claim 27, wherein the detection mechanism comprises a bright-field microscope.

32. The mDNB system of claim 27, wherein the NPs comprise metallic nanoparticles (MNPs).

33. The mDNB system of claim 27, wherein a microfluidic chip or cartridge comprises the mDNB device.

34. The mDNB system of claim 27, wherein the system comprises a point-of-care device that comprises or is configured to receive the mDNB device.

35. The mDNB system of claim 27, wherein the first recognition moiety is a compound selected from the group consisting of: biotin, streptavidin, avidin, an antibody, an antigen, an aptamer, a protein, a peptide, and a carbohydrate.

36. The mDNB system of claim 27, wherein the second recognition moiety is a compound selected from the group consisting of: biotin, streptavidin, avidin, an antibody, an antigen, an aptamer, a protein, a peptide, and a carbohydrate.

37. The mDNB system of claim 27, wherein the mDNB system is configured to detect the NT-proBNP molecules in a range of about 1-10,000 pg/mL from less than about 10 μL of a given whole blood sample in about 15 minutes or less.
